(19) United States
(12) Patent Application Publication
(10) Pub. No.: US 2026/0215936 A1
(43) Pub. Date: Jul. 30, 2026

(54) FOOT AND ANKLE PROSTHESIS

(71) Applicant: UNIVERSIDAD AUTÓNOMA DEL ESTADO DE MÉXICO, Toluca (MX)

(72) Inventors: Joel ZAGOYA LÓPEZ, Toluca (MX); Luis Adrián ZÚÑIGA AVILÉS,, Toluca (MX); Adriana Herlinda VILCHIS GONZÁLEZ,, Toluca (MX); Juan Carlos ÁVILA VILCHIS, Toluca (MX)

(73) Assignee: UNIVERSIDAD AUTÓNOMA DEL ESTADO DE MÉXICO, Toluca (MX)

(21) Appl. No.: 19/150,706

(22) PCT Filed: Nov. 16, 2023
(86) PCT No.: PCT/MX2023/050066

§ 371 Date: Jul. 24, 2025

(30) Foreign Application Priority Data
Jan. 25, 2023 (MX) MX/A/2023/001078

(51) Int. Cl. A61F 2/66; A61F 2/50

(52) U.S. Cl. CPC A61F 2/6607; A61F 2002/5001; A61F 2002/5073; A61F 2002/5083; A61F 2002/6614

(57) ABSTRACT

The present invention relates to a foot and ankle prosthesis called Fpros21 that allows the foot to move in the plantar flexion/dorsiflexion and eversion/inversion directions and comprises four groups: a set of aesthetic covers, a connection group for connecting the socket to the leg, an ankle mechanism, and a prosthetic foot. Fpros21 can function in ordinary use mode with a range of foot motion following a gait cycle with 15° of dorsiflexion and −28° of plantar flexion, and in hyperflexion use mode with a range of foot motion, in particular with −35° of plantarflexion, to perform actions related to driving a automobile. The prosthesis is created in 11 steps, including the design and manufacture of its various components, as well as the respective tests that verify its proper functioning.

### TECHNICAL FIELD

The present invention relates to techniques used in mechatronics, robotics, mechanical, and biomedical engineering to design and construct apparatus and/or devices that comprise prostheses for replacement of lower limbs with energy return storage to be used in patients with transtibial amputation, used in research fields related to mechanisms that generate bending/extension and inversion/eversion movements of the ankle, thereby allowing patients to perform activities such as walking or driving.

### PURPOSE OF THE INVENTION

It is a device that mobilizes joints to be used as a multi-jointed structure as a prosthesis according to the modalities of use, such that the device, purpose of the invention, allows selection of the operation modes.

The present invention is intended to provide a foot and ankle prosthesis capable of emulating foot dorsiflexion, plantarflexion, eversion and inversion movements, by means of a carbon fiber foot, a central spring and two rear springs that control the eversion and inversion movements.

The present invention is further intended to provide a functional and esthetic covers configuration for the prosthesis.

The present invention is further intended to provide a configuration of a mechanism called an ankle to perform hyperflexion of the user's foot.

The present invention is further intended to provide two use modes of a prosthesis, the first being focused on automobile driving and the other on the action of walking.

The present invention is further intended to provide an arrangement constituting a prosthesis whose manufacturing process allows to obtain a low-maintenance prosthesis.

### BACKGROUND

The design of lower limb prostheses dates back to the beginning of civilization, the oldest one for which there is evidence corresponds to a partial foot prosthesis in ancient Egypt; since then, until almost modern times, prostheses were mostly aesthetic and not very functional due to technological limitations and scarce material developments. It was not until the mid 1950's with the development of the Solid Ankle-Cushion Heel (SACH)-foot that a system capable of absorbing impacts while emulating plantar bending was designed; despite the limitations of movement, variations of this prosthesis are currently used because of their low cost and reliability. Another prosthesis type that has been developed in recent years is that of energy storage and return, better known as Energy storing and return (ESAR), developed in the early 1980s. This type of prosthesis stores energy via flexible keel to later release it mechanically, providing the necessary impulse to initiate the gait cycle. Currently, ESAR prostheses are constructed with composite materials such as carbon fiber, which reduce energy loss while reducing the weight of the foot. A different way of energy storage and release is by means of controlled energy storage and return prosthesis or Controlled Energy Storing and Returning (CESR), which stores energy using a spring or actuator, which at the moment of making contact with the ground, maintains a compact shape with the help of electronic locks until they are released with the foot movement, allowing the spring to elongate, thus providing the necessary impulse. The latest prostheses developed are the so-called active prostheses that not only work based on energy storage, but with the help of an external source such as actuators or artificial muscles, provide better control and stability in a gait cycle.

United States of America, China and Japan are the 3 main countries conducting research in the prostheses field. Because of these developments, researchers from the Universidad Autónoma del Estado de México proposed the design and manufacture of the prosthesis called FPROS-21.

### BRIEF DESCRIPTION OF THE DRAWINGS

The novel aspects that are considered characteristic of the present invention are particularly set forth in the appended claims. However, the invention itself, both by its organization and by its method of operation, together with other objects and advantages thereof, are explained in greater detail in the following description of a particularly preferred embodiment of the present invention, when read in relation to the accompanying figures, in which:

FIG. 1 is an isometric view of a prosthesis showing the arrangement of its five aesthetic covers (1-5), as worn by the user.

FIG. 2 is rear perspective view without the aesthetic covers, where a set of mechanisms that comprise the prosthesis and a connection of the leg socket (6-8) can be seen.

FIG. 3 is a frontal isometric view of a prosthetic foot, where the plantarflexion and dorsiflexion mechanisms can be seen (9-15, 19), as well as the prosthetic foot comprised of 5 plates (16-18, 24 and 29), which allows walking on uneven terrain more easily.

FIG. 4 is rear isometric view, where a mechanism performing eversion and inversion of the foot (21-23, 25-28) and two prosthetic foot plates (24, 29) can be seen.

FIG. 5 shows a rotation mechanism (20, 23, 27 and 30) that allows dorsiflexion/plantarflexion and bending/eversion movement in two degrees of freedom.

FIG. 6 shows the connection of two rear springs (22, 28) that regulate the inversion/eversion movements of the prosthetic foot (20, 26).

FIG. 7 shows the connections of an ankle (31-36) to the rotation mechanism and the support section of a right main spring (32a) and the left main support section (31a).

FIG. 8 is a rear view that shows the operation of the foot inversion/eversion springs, being the displacement to the left (a), neutral (b) and right (c) side.

FIG. 9 shows a user wearing the prosthesis without aesthetic covers during a gait cycle (a-f).

FIG. 10 shows the operation and bending of the prosthetic foot during the gait cycle, as well as the springs compression during a gait cycle (a-f).

FIG. 11 shows the displacement of the aesthetic covers (1-5) as the prosthetic foot plates bend.

FIG. 12 shows the process, from design to the manufacture of the prosthesis.

### FIRST SECTION: GENERAL DESCRIPTION OF THE INVENTION

The present invention comprises a prosthesis called Fpros21 for restoring the user's ability to walk and drive automobiles based on foot dorsiflexion/plantarflexion and eversion/inversion movements, as well as its manufacturing process, in an effective and safe manner. The invention is developed as a different contribution to the known prostheses; as well as it allows to be used in two operation modes (ordinary and hyperflexion).

The following specifications, it should be understood that certain position assignments such as “over”, “forward”, “backward” are made with reference to the mobilizing device in normal use position with a horizontally arranged mobilizing axis. Backward and forward terms are used relatively to the mobilizing direction, just as downward and upward terms are used in relation to the referred axis.

Fpros21 is a human body-powered prosthesis that enables plantarflexion, dorsiflexion, eversion and inversion movements of an amputated foot. Said prosthesis comprises four groups: set of aesthetic covers, leg socket connection group, ankle mechanism and prosthetic foot.

### SECOND SECTION: DETAILED DESCRIPTION OF THE INVENTION

In designing and manufacturing prosthetic foot, the user's requirements, as well as functional and anatomical requirements, are compiled through research in scientific literature and the current state of foot prostheses. A combination of methodologies comprising design thinking, user-centered design and design for manufacturing is employed. From the data obtained, a quality function deployment (QFD) is performed to translate the requirements into design features, which comprise, for the anatomical part, movements in dorsiflexion of 15°, plantarflexion of −28°, eversion of 5°, inversion of −14°, internal rotation of −8°and external rotation of 15°. For the operational requirements part, it is defined that the prosthesis requires an ankle torque average of 124 Nm, ankle power of 281 W, mass of 3.67 kg, mainspring stiffness of 339 kNm, mainspring energy storage of 6.3 J, and duty cycle of 4000 steps per day.

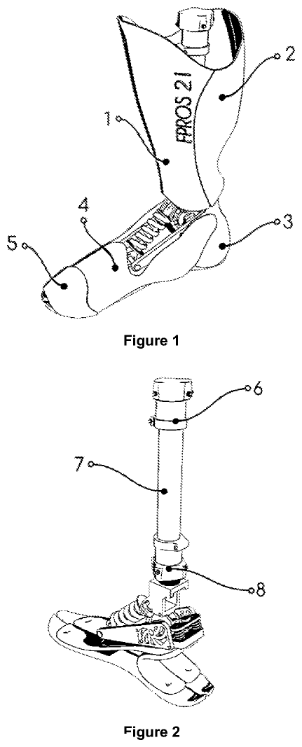

A set of aesthetic covers (FIG. 1) is comprised of two ankle covers (1 and 2), heel cover (3), foot cover (4) and toe cover (5). These covers provide better visual appeal, protect the prosthesis and are designed not to restrict foot movement. The function of the esthetic covers is to increase the user's empathy with the prosthesis while at the same time protecting the different mechanisms and allowing the use of footwear over the prosthesis.

A leg socket connection group comprises two connectors (6 and 8) and an aluminum rod (7) that connects the prosthesis to the amputated limb, which are fastened by means of clamps.

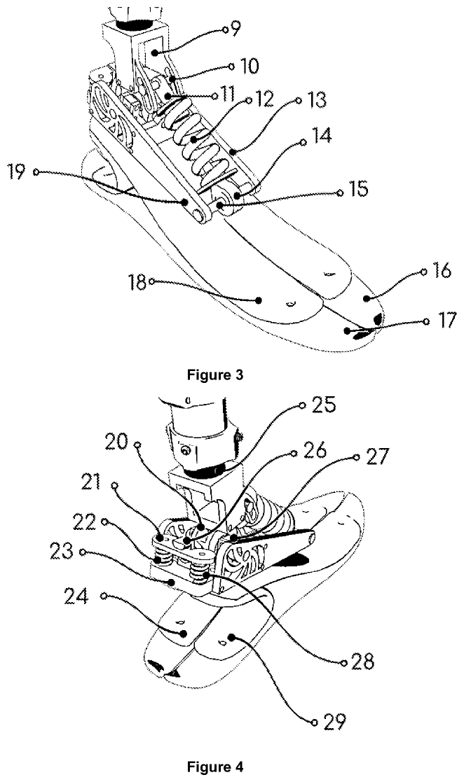

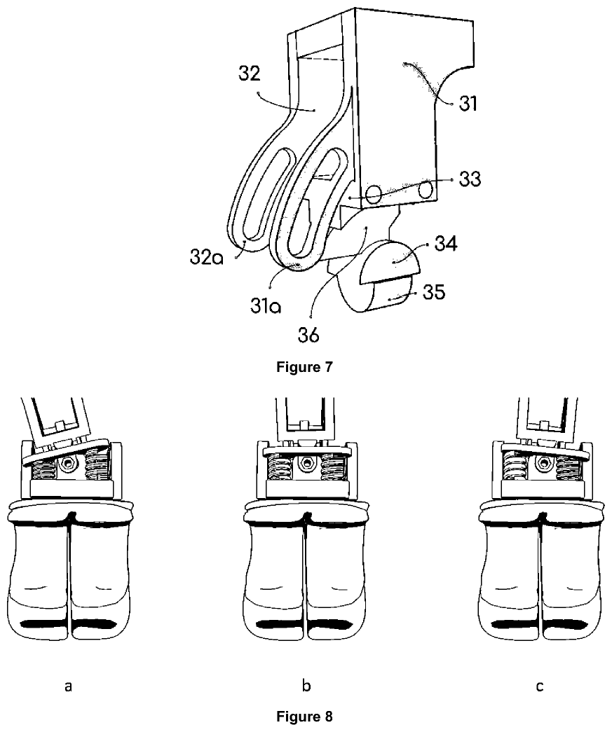

An ankle mechanism (FIGS. 3-7) is comprised of a foot inversion/eversion mechanism and 23 components (9-19, 20-23, 25-28 and 31-36). Said components comprise: two grooved plates (31 and 32) that allow the sliding of the shaft (10), a base (33), two semicircular pieces (34 and 35) that are coupled to the shafts 20 and 27 to allow a circular movement of the foot, and are connected to the base (33) by a metal piece (36) whose shape restricts the dorsiflexion and plantarflexion movement; also has a main spring (12) in charge of storing/releasing the energy necessary to initiate a gait cycle, and two rear springs (22, 28) that are in charge of the inversion/eversion movements of the prosthetic foot. As part of the ankle mechanism, the shaft (10) is connected to a bracket (11) and to the main spring (12) which is connected to a bearing (14) that adjusts to the required angle during plantarflexion and dorsiflexion movements, and in turn is connected to a shaft (15). The spring system is joined to the rotation mechanism (20) by two plates (13 and 19), which support the stresses generated during the gait cycle.

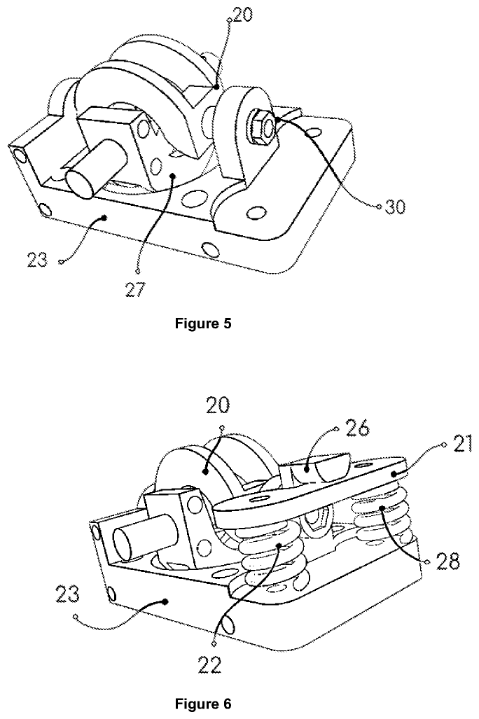

The foot inversion/eversion mechanism (FIG. 5) is in charge of providing movements of 5° for eversion (8a) and −14° in inversion (8c) and is comprised of two shafts (20 and 27), these shafts are connected to the base and by means of a connector (30) joins the springs (22 and 28) that provide a return of the foot to the initial position (8b). The plate (21) together with piece (26) are connected to the shaft (20) and are responsible for exerting pressure on the springs (22 and 28). The shaft (20) is responsible for providing plantarflexion and dorsiflexion movements by displacing the ankle between 15° and −28°.

A prosthetic foot is made up of five carbon fiber plates (16-18, 24 and 29) divided to adapt to uneven terrain, which allow for energy storage and return, as well as emulating the foot inversion and eversion movements.

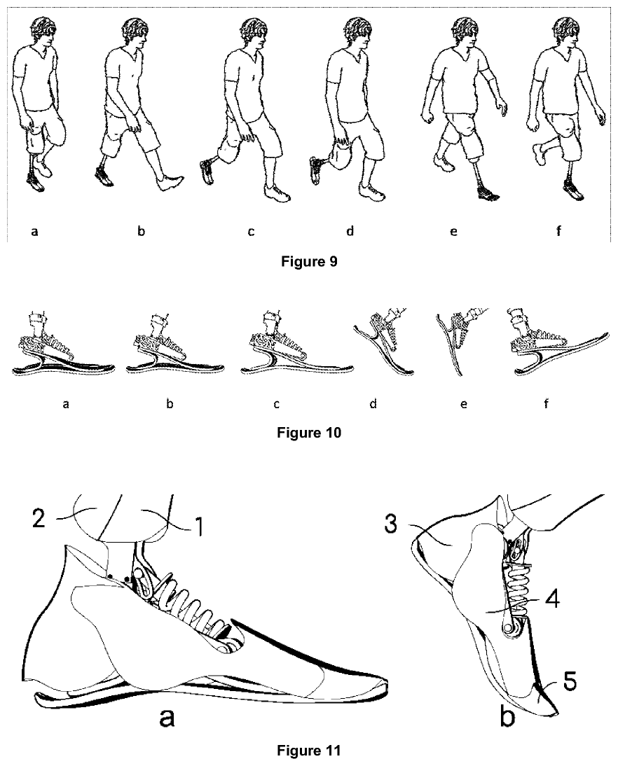

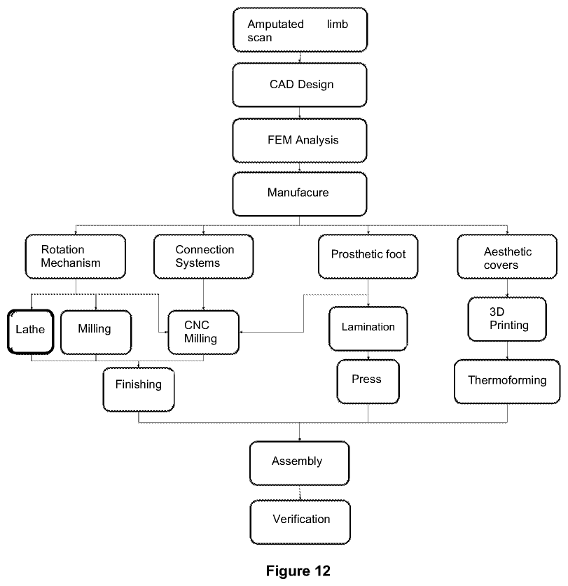

In the gait cycle and during the initial contact phase, the heel comes into contact with the ground by bending the plates (10e) until the load response phase (10f), during intermediate support, the plantarflexion and dorsiflexion spring begins energy storage (10a and 10b), during the final support phase, the spring reaches maximum energy storage (10c), as well as the foot carbon fiber plates. During the initial stage of the swing phase, the energy of the spring and plates is released (10d) generating the impulse necessary to complete the gait cycle, (FIGS. 9 and 10).

During the gait cycle and to allow the aforementioned movements, the aesthetic covers are accommodated following the foot bending (FIG. 11).

The ordinary use mode comprises the range of foot motion during a gait cycle, 15° of dorsiflexion and −28° of plantarflexion. The hyperflexion use mode comprises the range of foot motion for automobile driving functions, being −35° of plantarflexion.

The FPROS21 prosthesis manufacturing process is comprised of 11 steps, which are described from step 1 to 4 sequentially and from step 5 to 10 concurrently, while step 11 refers to the verification of the prosthesis according to a test protocol.

The first step in designing FPROS21 is to obtain the appropriate measurements for the patient, so a 3D scan of the user is performed with the precise measurements of the amputated limb.

In the second step, a CAD model of the mechanical elements is made according to the scan of the previous step to ensure that the measurements and volume of the prosthesis are suitable for the user.

As a third step, all the system pieces are analyzed using Finite Element Method (FEM) to validate and stability of all the mechanisms of the prosthesis before manufacturing it.

Subsequently for its manufacture and as a fourth step. it is considered that the model is replicable, so machines commonly found in manufacturing laboratories are chosen, which include milling machine, CNC milling machine, lathe, drill, press, 3D printer and thermoforming. Likewise, components used in similar devices (commonality index) are analyzed for use in the product family using the formula CC=(100*components in common)/(components in common-unique components), resulting in 91% of components in common.

As a fifth step, the bases and supports of the mechanisms are manufactured, such as the side plates (19, 13), from a CNC milling machine on a 5 mm aluminum plate, the base (23) from a 3 mm thick aluminum block on CNC, the details are refined on a milling machine, the holes are drilled and the holes thread for the lomilleries are made. The ankle base (33) is made in stainless steel of 30 mm thickness on a milling machine and holes are drilled in the sides in a round stainless-steel profile, the circular pieces (34 and 35) are turned and trimmed with a milling machine, drilled and joined to the base (33) by means of an M3 screw. Plates 31 and 32 are CNC-fabricated from 5 mm thick aluminum plates, the pieces are drilled and joined to the base to form the ankle mechanism.

As a sixth step, the components of the different mechanisms are manufactured, such as the semicircular plates (20, 27), from a 5 mm thick aluminum plate, which are milled down to 2.5 mm and CNC cut. The shafts of the rotation mechanism are made of 5 mm blocks of stainless steel; firstly, in a milling machine and then turned on a lathe. The pieces are then assembled with M3 screws to form the rotation shafts (30). Pieces 26 and 21 are CNC machined from aluminum, drilled and the thread for M2 screws is made.

As a seventh step, the components that join the bases to the mechanisms are made, for the shafts (15 and 10) using 5 mm thick round stainless-steel profiles by means of a lathe. The bushing (11) is made of a stainless-steel cylinder and milled to obtain a shape that fits the shaft (10).

As an eighth step, the aesthetic covers (1-5) are produced by 3D printing with a coating obtained from thermoforming to increase strength and improve the finish of the 3D print.

In the ninth step, the prosthetic foot (16-18, 24 and 29) is manufactured with 3K carbon fiber fabric and epoxy resin, for this, 12 molds (male and female) are made in CNC machine to obtain each of the plates of the foot, the fabric already cut is placed in the mold's shape and a lamination process is performed until a thickness of 6 mm is achieved, the molds are pressed to compact the plates and finally they are assembled using Alien type screws.

In the tenth step, the pieces (20 and 27) are assembled onto the ankle (9) and placed onto the base (23), then the side pieces (13, 19) are joined, the shafts (10, 15) are placed into the bearings (11 and 14) together with the main spring (12). The pieces (26 and 21) are placed together with the springs (22 and 28). The ankle mechanism already assembled is joined to the prosthetic foot using M8 screws and finally the aesthetic covers are press-fitted.

In the eleventh step, the prosthesis is verified according to a test protocol that evaluates the system in its two of operation modes using a test bench that allows to verify the data obtained from the requirements, which comprise, for the anatomical part, movements 15° of dorsiflexion, −28° of plantarflexion, 5° of eversion, −14° of inversion, −8° of internal rotation and 15° of external rotation. For the operational requirements part, it is defined that the prosthesis requires an ankle torque average of 124 NM, ankle power of 281 W, mass of 3.67 kg, mainspring stiffness of 339 kNm, mainspring energy storage of 6.3 J, and duty cycle of 4000 steps per day.

Having sufficiently described the invention, the following is considered novel and is therefore claimed as exclusive property:

1. A lower limb prosthesis for the foot and ankle composed of 36 components and comprising:
a set of aesthetic covers, where two of them (1,2) are press-coupled to a leg socket connection group (6-8), and three more (3-5) are coupled to both an ankle mechanism and a prosthetic foot;
said leg socket connection group (6-8) is assembled to the ankle mechanism, by means of four clamps that keep the prosthesis connector (8) joined to the pyramidal connector (25);
likewise, the ankle mechanism is connected to the prosthetic foot by means of M8 screws that join a foot plate (18) with a base (23) of the ankle mechanism;
characterized in that the prosthesis mechanisms allow the foot inversion/eversion movement between 5° and −14 ° with respect to the main shaft (30);
likewise, the prosthesis can be configured to be worn either the right or left side of a user by angularly displacing a rotation mechanism of the ankle mechanism by 180°;
as well as said prosthesis can be used in two use modes, ordinary and hyperflexion and can be adapted to different users by replacing the main spring (12) and rear springs (22 and 28).

2. The lower limb prosthesis for the foot and ankle according to claim 1, characterized in that the set of aesthetic covers is comprised of two ankle covers (1 and 2), heel cover (3), foot cover (4) and toe cover (5); these covers provide better visual appeal, protect the prosthesis and are designed not to restrict foot movement;
the function of the esthetic covers is to increase the user's empathy with the prosthesis while at the same time protecting the different mechanisms and allowing the use of footwear over the prosthesis.

3. The lower limb prosthesis for the foot and ankle according to claim 1, characterized in that the leg socket connection group comprises two connectors (6 and 8) and an aluminum rod (7) that connects the prosthesis to the amputated limb, which are fastened by means of eight clamps.

4. The lower limb prosthesis for the foot and ankle according to claim 1, characterized in that the ankle mechanism is comprised of a foot inversion/eversion mechanism and 23 components (9-19, 20-23, 25-28 and 31-36);
said components comprise:
two grooved plates (31 and 32) that allow the sliding of the shaft (10), a base (33) two semicircular pieces (34 and 35) that are coupled to the shafts 20 and 27 to allow a circular movement of the foot, and are connected to the base (33) by a metal piece (36) whose shape restricts the dorsiflexion and plantarflexion movement;
also has a main spring (12) in charge of storing/releasing the energy necessary to initiate a gait cycle, and two rear springs (22,28) that are in charge of the inversion/eversion movements of the prosthetic foot;
the shaft (10) is connected to a bracket (11) and to the main spring (12) which is connected to a bearing (14) that adjusts to the required angle during plantarflexion and dorsiflexion movements, and in turn is connected to a shaft (15);
the spring system is joined to the rotation mechanism (20) by two plates (13 and 19), which support the stresses generated during the gait cycle.

5. The lower limb prosthesis for the foot and ankle according to claim 1, characterized in that the foot inversion/eversion mechanism is in charge of providing movements of 5° for eversion and −14° in inversion and is comprised of two shafts (20 and 27), these shafts are connected to the base and by means of a connector (30) joins the springs (22 and 28) that provide a return of the foot to the initial position (8b);
the plate (21) together with piece (26) are connected to the shaft (20) and are responsible for exerting pressure on the springs (22 and 28);
the shaft (20) is responsible for providing plantarflexion and dorsiflexion movements by displacing the ankle between 15° and −28°.

6. The lower limb prosthesis for the foot and ankle according to claim 1, characterized in that the prosthetic foot is made up of five carbon fiber plates (16-18, 24 and 29) divided to adapt to uneven terrain, which allow for energy storage and return, as well as emulating the foot's inversion and eversion movements.

7. The lower limb prosthesis for the foot and ankle according to claim 1, characterized in that in the gait cycle and during the initial contact phase, the heel comes into contact with the ground by bending the plates (10e) until the load response phase (10f), during intermediate support, the plantarflexion and dorsiflexion spring begins energy storage (10a and 10b), during the final support phase, the spring reaches maximum energy storage (10c), as well as the foot carbon fiber plates;
during the initial stage of the swing phase, the energy of the spring and plates is released (10d) generating the impulse necessary to complete the gait cycle.

8. The lower limb prosthesis for the foot and ankle according to claim 1, characterized in that during the gait cycle and to allow the aforementioned movements, the aesthetic covers are accommodated following the foot bending.

9. The lower limb prosthesis for the foot and ankle according to claim 1, characterized in that the ordinary use mode comprises the range of foot motion during a gait cycle, 15° of dorsiflexion and −28° of plantarflexion;
the hyperflexion use mode comprises the range of foot motion for automobile driving functions, being −35° of plantarflexion.

10. A lower limb prosthesis for the foot and ankle, characterized in that the FPROS21 prosthesis manufacturing process is comprised of 11 steps, which are described from step 1 to 4 sequentially, from step 5 to 10 concurrently, and eleventh step refers to the verification of the prosthesis according to a test protocol.

11. The lower limb prosthesis for the foot and ankle according to claim 10, characterized in that the first step in designing FPROS21 is to obtain the appropriate measurements for the patient, so a 3D scan of the user is performed with the precise measurements of the amputated limb.

12. The lower limb prosthesis for the foot and ankle according to claim 10, characterized in that in the second step, a CAD model of the mechanical elements is made according to the scan of the previous step to ensure that the measurements and volume of the prosthesis are suitable for the user.

13. The lower limb prosthesis for the foot and ankle according to claim 10, characterized in that in the third step, all the system pieces are analyzed using Finite Element Method (FEM) to validate and stability of all the mechanisms of the prosthesis before manufacturing it.

14. The lower limb prosthesis for the foot and ankle according to claim 10, characterized in that for its manufacturing, the model is replicable, so machines commonly found in manufacturing laboratories are chosen, which include milling machine, CNC milling machine, lathe, drill, press, 3D printer and thermoforming;
likewise, components used in similar devices (commonality index) are analyzed for use in the product family using the formula CC=(100*components in common)/(components in common-unique components), resulting in 91% of components in common.

15. The lower limb prosthesis for the foot and ankle according to claim 10, characterized in that in the fifth step, the bases and supports of the mechanisms are manufactured, such as the side plates (19,13), from a CNC milling machine on a 5 mm aluminum plate, the base (23) from a 3 mm thick aluminum block on CNC, the details are refined on a milling machine, the holes are drilled and the holes thread for the lomilleries are made;
the ankle base (33) is made in stainless steel of 30 mm thickness on a milling machine and holes are drilled in the sides in a round stainless-steel profile, the circular pieces (34 and 35) are turned and trimmed with a milling machine, drilled and joined to the base (33) by means of an M3 screw;
plates 31 and 32 are CNC-fabricated from 5 mm thick aluminum plates, the pieces are drilled and attached to the base to form the ankle mechanism.

16. The lower limb prosthesis for the foot and ankle according to claim 10, characterized in that in the sixth step, the components of the different mechanisms are manufactured, such as the semicircular plates (20,27), from a 5 mm thick aluminum plate, which are milled down to 2.5 mm and CNC cut;
the shafts of the rotation mechanism are made of 5 mm blocks of stainless steel;
firstly, in a milling machine and then turned on a lathe; the pieces are then assembled with M3 screws to form the rotation shafts (30);
pieces 26 and 21 are CNC machined from aluminum, drilled and the thread for M2 screws is made.

17. The lower limb prosthesis for the foot and ankle according to claim 10, characterized in that in the seventh step, the components that join the bases to the mechanisms are made, for the shafts (15 and 10) using 5 mm thick round stainless-steel profiles by means of a lathe;
the bushing (11) is made of a stainless-steel cylinder and milled to obtain a shape that fits the shaft (10).

18. The lower limb prosthesis for the foot and ankle according to claim 10, characterized in that in the eighth step, the aesthetic covers (1-5) are produced by 3D printing with a coating obtained from thermoforming to increase strength and improve the finish of the 3D print.

19. The lower limb prosthesis for the foot and ankle according to claim 10, characterized in that in the ninth step, the prosthetic foot (16-18, 24 and 29) is manufactured with 3K carbon fiber fabric and epoxy resin, for this, 12 molds (male and female) are made in CNC machine to obtain each of the plates of the foot, the fabric already cut is placed in the mold's shape and a lamination process is performed until a thickness of 6 mm is achieved, the molds are pressed to compact the plates and finally they are assembled using Alien type screws.

20. The lower limb prosthesis for the foot and ankle according to claim 10, characterized in that in the tenth step, the pieces (20 and 27) are assembled onto the ankle (9) and placed onto the base (23), then the side pieces (13,19) are joined, the shafts (10,15) are placed into the bearings (11 and 14) together with the main spring (12); the pieces (26 and 21) are placed together with the springs (22 and 28); the ankle mechanism already assembled is joined to the prosthetic foot using M8 screws and finally the aesthetic covers are press-fitted.

21. The lower limb prosthesis for the foot and ankle according to claim 10, characterized in that in the eleventh step, the prosthesis is verified according to a test protocol that evaluates the system in its two operation modes using a test bench that allows to verify the data obtained from the requirements, which comprise, for the anatomical part, movements 15° of dorsiflexion, −28° of plantarflexion, 5° of eversion, −14° of inversion, −8° of internal rotation and 15° of external rotation; for the operational requirements part, it is defined that the prosthesis requires an ankle torque average of 124 NM, ankle power of 281 W, mass of 3.67 kg, mainspring stiffness of 339 kNm, mainspring energy storage of 6.3 J, and duty cycle of 4000 steps per day.
